(19) United States
(12) Patent Application Publication
(10) Pub. No.: US 2006/0097879 A1
(43) Pub. Date: May 11, 2006

(54) SIDS and apnea monitoring system

(76) Inventor: Kathy J. Lippincott, Gresham, OR (US)

Correspondence Address: JOSEPH P. CURTIN, 1469 N.W. MORGAN LANE, PORTLAND, OR, 97229, US

(21) Appl. No.: 10/973,223

(22) Filed: Oct. 26, 2004

(51) Int. Cl. G08B 23/00; A61B 5/00; G08B 1/08

(52) U.S. Cl. 340/573.1; 340/539.12; 600/300

(57) ABSTRACT

A sleeping person is monitored by a system having a sensor unit and a base unit. The sensor unit outputs a sensor signal corresponding to each of at least two body function parameters of a sleeping person, such as a heart-rate, a respiratory rate and/or a body temperature of the sleeping person. The base unit determines whether an alarm condition is present based on each sensor signal output from the sensor unit and, in response to a detected alarm condition, generating an actuation signal for actuating an audible signal, such as a recording of a voice of a caregiver, in proximity of the sleeping person. The sensor unit also includes a shock-delivering device, and the base unit generates an actuation signal for actuating the shock-delivering device for stimulating the sleeping person to do one of breathe and enter a less deep sleep pattern.

### BACKGROUND OF THE INVENTION

1. Field of the Invention

The present invention relates to an apnea monitoring system. More particularly, the present invention relates to a system and a method for monitoring conditions of a sleeping person.

2. Description of the Related Art

Sudden Infant Death Syndrome-type (SIDS-type) monitors are known. For example, U.S. Patent Application No. 2002/0057202 A1 to Luzon discloses an infant monitoring system that functions as a communication link between an infant lying in, for example, a crib and, for example, the infant's mother at a remote (from the infant) monitoring station. The Luzon monitoring system alerts the mother to any one of three vital conditions, specifically, a breathing condition, a fever condition and a crying condition, that require immediate attention.

Another example of a SIDS-type monitor is U.S. Patent Application No. 2002/0097155 A1 to Cassel et al., which discloses a combination audio baby alarm and breathing monitor. The audio alarm portion monitors sounds within a baby's environment and transmits the sounds for reproduction at a receiver. The breathing monitor portion identifies alarmable events in the breathing of the infant or heart rate and generates an alarm annunciator signal that is transmitted for reproduction at the same receiver.

While both of these exemplary SIDS-types monitors and other similar SIDS-types monitors alert someone who is remotely located from an infant detected to be in SIDS-type distress, the time taken for someone to physically respond to such an alarm may be too long for the infant. Consequently, what is needed is a way to attempt to revive an infant in SIDS-type distress while someone is physically responding.

### BRIEF SUMMARY OF THE INVENTION

The present invention provides a way to attempt to revive an infant in SIDS-type distress while someone is physically responding.

The advantages of the present invention are provided by a system including a sensor unit and a base unit. The sensor unit outputs a sensor signal corresponding to each of at least two body function parameters of a sleeping person. For example the sensor unit outputs a sensor signal corresponding to a heart-rate, a respiratory rate and/or a body temperature of the sleeping person. The base unit determines whether an alarm condition is present based on each sensor signal output from the sensor unit and, in response to a detected alarm condition, generating an actuation signal for actuating an audible signal, such as a recording of a voice of a caregiver, in proximity of the sleeping person. The detected alarm condition includes at least one of a heart rate lower than a predetermined heart rate, a respiratory rate that is lower than a predetermined respiratory rate, a body temperature that is less than a first predetermined body temperature and a body temperature that is greater than a second predetermined body temperature. The sensor unit also includes a shock-delivering device, and the base unit generates an actuation signal for actuating the shock-delivering device for stimulating the sleeping person to do one of breathe and enter a less deep sleep pattern. The system also includes a monitoring unit having a display and an audible actuator. The base unit sends a monitoring signal representing each sensor signal output by the sensor unit to the monitoring unit, and the display on the monitoring unit, in response to the monitoring signal, displays a current value of each sensor signal. The base unit can also have a display displaying a current value of each sensor signal.

The present invention also provides a method of monitoring a sleeping person, in which a sensor signal is generated corresponding to each of at least two body function parameters of a sleeping person. The sensor signal can correspond to, for example, a heart-rate, a respiratory rate and/or a body temperature of the sleeping person. It is then determined whether an alarm condition is present based on each sensor signal output from the sensor unit. The detected alarm condition includes at least one of a heart rate lower than a predetermined heart rate, a respiratory rate that is lower than a predetermined respiratory rate, a body temperature that is less than a first predetermined body temperature and a body temperature that is greater than a second predetermined body temperature. An actuation signal is generated in response to a detected alarm condition for actuating an audible signal, such as a recording of a voice of a caregiver, in proximity of the sleeping person. An actuation signal can also be generated for actuating a shock-delivering device for stimulating the sleeping person to do one of breathe and enter a less deep sleep pattern. The current value of each sensor signal can also be displayed.

### BRIEF DESCRIPTION OF THE DRAWINGS

The present invention is illustrated by way of example and not by limitation in the accompanying figures in which like reference numerals indicate similar elements and in which:

FIG. 1 depicts a functional block diagram of a sensor unit according to the present invention that includes sensors for monitoring body functions of a sleeping person;

FIG. 2 depicts a functional block diagram of a base unit according to the present invention that processes sensor signals received from the sensor unit depicted in FIG. 1; and

FIG. 3 depicts a functional block diagram of a monitoring unit according to the present invention that can be worn by a caregiver.

### DETAILED DESCRIPTION OF THE INVENTION

The present invention provides a monitoring system that can be used for monitoring babies, children and adults who exhibit signs of sleeping disorders, such as Sudden Infant Death Syndrome (SIDS), apnea and/or narcolepsy.

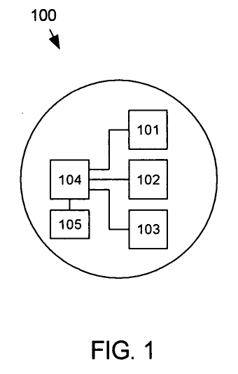

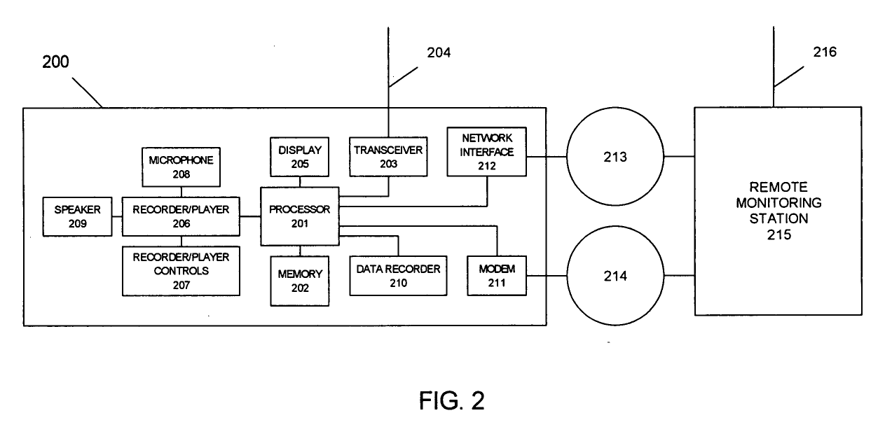

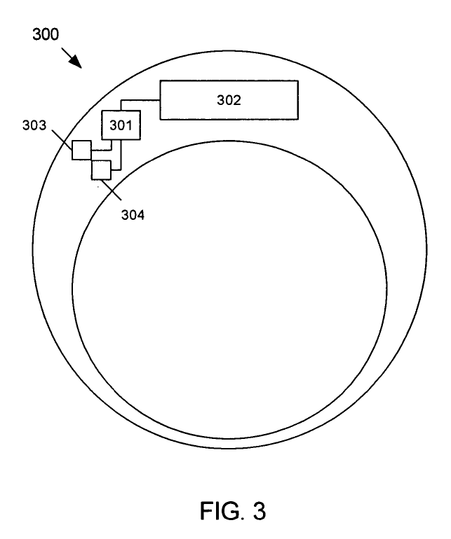

FIGS. 1-3 depict block diagrams of the functional units of the present invention. In particular, FIG. 1 depicts a functional block diagram of a sensor unit 100 that includes sensors for monitoring body functions of a sleeping person. FIG. 2 depicts a functional block diagram of a base unit 200 that processes sensor signals received from sensor unit 100 and transmits monitoring information to a remote station 215 and/or a monitoring unit 300. FIG. 3 depicts a functional block diagram of a monitoring unit 300 that can be worn by a caregiver.

In FIG. 1, sensor unit 100 includes a heart-rate sensor 101, a breathing sensor 102, a body temperature sensor 103, a transceiver 104 and a shock-delivering device 105. Heart-rate sensor 101, breathing sensor 102 and body temperature sensor 103 are each coupled to a transceiver 104 that transmits sensor information relating to a heart rate signal, a breathing signal and a body temperature signal, respectively, to a base unit 200, which is shown in FIG. 2, in a well-known manner, using an antenna (not shown) and a radio-frequency (rf) link (not shown). Each of heart-rate sensor 101, breathing sensor 102 and body temperature sensor 103 are well-known sensors that each operate in a well-known manner. Transceiver unit 104 receives an actuation signal from base unit 200 and, in response to the actuation signal, causes a shock-delivering device 105, such as a piezoelectric element, to deliver a mild electric shock to stimulate a sleeping person to breathe or to bring a detected deep-sleep pattern to be less deep sleep.

Sensor unit 100 is disposed in close proximate relation to a person who is to be monitored, for example, in direct contact with the skin in the chest region of a sleeping person. Alternatively, sensor unit 100 can be attached in a well-known manner to an article of clothing that the person is wearing. Base unit 200 is also placed in a proximate relationship to the person who is to be monitored, such as attached to the head of a crib or bed, placed on a night table next to a bed or on a shelf in the same room as the person who is to be monitored.

Base unit 200, depicted in FIG. 2, includes a processor 201, a memory 202, a transceiver 203 having an antenna 204, a display 205, a recorder/player 206, manual controls 207 for recorder/player 206, a microphone 208, a speaker 209. Additionally, base unit 200 can include a data recorder 210, a modem 211 and/or a network interface 212. Processor 201 is coupled to memory 202, transceiver 203, display 204, recorder/player 205, data recording unit 210, modem 211 and network interface 212 in a well-known manner. Transceiver 203 receives sensor signals that have been transmitted by sensor unit 100, and passes the sensor signals to processor 201. Processor 201 monitors the different sensor signals that have been received from sensor unit 100 and, in a well-known manner, compares the respective values of the sensor signals to corresponding alarm values stored in memory 202. Processor 201 can store the received sensor information in data recording unit 210 for later retrieval and evaluation. For example, data recording unit 210 can be a mass storage device, such as a disk drive, an optical drive or a random access memory (RAM) card. Alternatively, processor 201 can also store the received sensor information in memory 202 for later retrieval and evaluation.

Processor 201 outputs the current monitoring conditions to display 204, which displays the heart rate, the respiration rate and the body temperature of the monitored person. Display 204 can also display information such as monitored maximum and minimum values of the heart rate, respiration rate and body temperature, the current time and other monitored information. While display 204 is preferably as a single Liquid Crystal Display (LCD) that displays all of the monitored parameters, display 204 could alternatively display the monitored information in a cyclic manner, thereby reducing the oversize of the display. It should also be understood that display 204 could alternatively be embodied as several separate displays. Additionally or as yet another alternative, display 204 could include lights or icons having selected shapes, such as a heart, a square and/or a circle, etc., that are actuated by processor 201 when alarm conditions are detected.

Processor 201 sends the processed monitoring information to remote monitoring station 215 through modem 211 and telecommunications network 214 in a well-known manner for recording and evaluation. Remote monitoring station 215 could be, for example, a caregivers' station in a hospital or care facility or a 911 center. Remote monitoring station 215 could be located within the same building as the sleeping person. Alternatively, remote monitoring station 215 could be located at another facility that is different from the location of the sleeping person. Telecommunications network 214 can be, for example, a Public-Switched Telephone Network (PSTN) or a wireless telecommunications network, such as a cellular telephone network or a Personal Communication System (PCS) network. Alternatively, processor 201 can send the processing monitoring information to remote monitoring station 215 through network interface 212 and telecommunications network 213, such as a Local Area Network (LAN), a Wide Area Network (WAN) or the Internet, in a well-known manner. As yet another alternative, processor 201 can send the processed monitoring information to remote monitoring station 215 through transceiver 203 and antenna 204, over a wireless link to an antenna 216 of remote monitoring station 215.

Processor 201 also sends the processed monitoring information to transceiver 203 for transmission to monitoring unit 300, which is depicted in FIG. 3 as a wrist band that is worn by a caregiver to the person being monitored. Alternatively, monitoring unit 300 could be configured to be attached to the clothing of a caregiver. Transceiver 203 has a range of several hundred feet to communicate with monitoring unit 300 so that a caregiver has some latitude in movement in and around, for example, a home where the monitored person is sleeping. (When base unit 200 is coupled to remote monitoring station 215 through a wireless link, transceiver 203 has sufficient output power to reliably communicate with remote monitoring station 215.) Monitoring unit 300 includes a receiver/processor 301, a display 302, an audible/vibratory alarm device 303, and/or a shock-delivering device 304. The processed monitoring information is received by an antenna (not shown) coupled to receiver/processor 301, receiver/processor 301 processes the monitoring information in a well-known manner. The received monitoring information is displayed on display 302, which is, for example, an LCD. Additionally, display 302 can include a light or an icon shaped like a heart that pulses, or flashes, at the heart rate of the sleeping person. Audible/vibratory alarm device 303 outputs an audible and/or a vibratory alarm when an alarm signal is received from base unit 200. Shock-delivering device 304 delivers a mild electric shock to alert a caregiver who may be sleeping.

When processor 201 determines that a received sensor signal is in an alarm condition, such as when the received heart-rate sensor signal has a rate that is below than the lower limit heart-rate value stored in memory 202, processor does at least two operations. For one operation, processor 201 transmits an alarm signal to monitoring unit 300 so that the caregiver can be alerted to the detected alarm condition. Monitoring unit 300 responds to the alarm signal by causing audible/vibratory alarm device 303 to produce an audible and/or a vibratory alarm and/or for shock-delivering device 304 to deliver a mild electric shock. Display 302 can additionally display information relating to the detected alarm condition. Alternatively or additionally, processor 201 causes a telephone call to be placed to a predetermined telephone number, such as the mobile telephone of a caregiver or to an emergency response center or remote station 215.

For the second operation, processor 201 transmits an actuation signal to recorder/player 206 that causes recorder/player 206 to output through speaker 209 an audible signal, such as a recording of a caregiver, such as a mother or a father, saying the name of the person being monitored. The audible signal can be manually recorded in a well-known manner by using manual controls 207 and microphone 208 and stored in, for example, memory 202. Alternatively, the audible signal could be stored on a non-volatile medium, such as a magnetic tape or a compact disc (CD). Alternatively or in addition, processor 201 sends an actuation signal to sensor unit 100 that causes shock-delivering device 105 to deliver a mild electric shock to stimulate a sleeping person to breathe or to bring a detected deep-sleep pattern to be less deep sleep. The actuation signal transmitted to sensor unit 100 can be transmitted repeatedly until the alarm condition is not detected any longer.

For the third operation, remote monitoring station 215 is alerted of an alarm condition simultaneously when either of the first two operations is performed.

The various components forming sensor unit 100, base unit 200 and monitoring unit 300 are well-known components.

Although the foregoing invention has been described in some detail for purposes of clarity of understanding, it will be apparent that certain changes and modifications may be practiced that are within the scope of the appended claims. Accordingly, the present embodiments are to be considered as illustrative and not restrictive, and the invention is not to be limited to the details given herein, but may be modified within the scope and equivalents of the appended claims.

What is claimed is:

1. A system, comprising:
a sensor unit outputting a sensor signal corresponding to each of at least two body function parameters of a sleeping person; and
a base unit determining whether an alarm condition is present based on each sensor signal output from the sensor unit and, in response to a detected alarm condition, generating an actuation signal for actuating a shock-delivering device for stimulating the sleeping person to do one of breathe and enter a less deep sleep pattern.

2. The system according to claim 1, wherein the base unit generates an actuation signal, in response to the detected alarm condition, for actuating an audible signal in proximity of the sleeping person.

3. The system according to claim 2, wherein the audible signal is a recording of a voice of a caregiver.

4. The system according to claim 1, wherein the sensor unit outputs a sensor signal corresponding to a heart-rate and a respiratory rate of the sleeping person.

5. The system according to claim 4, wherein the sensor unit further outputs a sensor signal corresponding to a body temperature of the sleeping person.

6. The system according to claim 1, wherein the detected alarm condition includes at least one of a heart rate lower than a predetermined heart rate, a respiratory rate that is lower than a predetermined respiratory rate, a body temperature that is less than a first predetermined body temperature and a body temperature that is greater than a second predetermined body temperature.

7. The system according to claim 1, further comprising a monitoring unit including a display and an audible actuator, and
wherein the base unit sends a monitoring signal representing each sensor signal output by the sensor unit to the monitoring unit, and
wherein the display on the monitoring unit, in response to the monitoring signal, displays a current value of each sensor signal.

8. The system according to claim 1, wherein the base unit further comprises a display displaying a current value of each sensor signal.

9. The system according to claim 1, wherein the base unit further comprises a heart-shaped indicator that indicates a heart rate of the sleeping person.

10. A method of monitoring a sleeping person, the method comprising:
generating a sensor signal corresponding to each of at least two body function parameters of a sleeping person;
determining whether an alarm condition is present based on each sensor signal output from the sensor unit; and
generating an actuation signal in response to a detected alarm condition for actuating a shock-delivering device for stimulating the sleeping person to do one of breathe and enter a less deep sleep pattern.

11. The method according to claim 10, further comprising generating an actuation signal, in response to the detected alarm condition, for actuating an audible signal in proximity of the sleeping person.

12. The method according to claim 11, wherein the audible signal is a recording of a voice of a caregiver.

13. The method according to claim 10, further comprising outputting a sensor signal corresponding to a heart-rate and a respiratory rate of the sleeping person.

14. The method according to claim 13, further comprising outputting a sensor signal corresponding to a body temperature of the sleeping person.

15. The method according to claim 10, wherein the detected alarm condition includes at least one of a heart rate lower than a predetermined heart rate, a respiratory rate that is lower than a predetermined respiratory rate, a body temperature that is less than a first predetermined body temperature and a body temperature that is greater than a second predetermined body temperature.

16. The method according to claim 10, wherein the audible signal is a recording of a voice of a caregiver.

17. The method according to claim 10, further comprising displaying a current value of each sensor signal.

18. The method according to claim 10, further comprising displaying a heart rate of the sleeping person using a heart-shaped indicator.
